(19) United States
(12) Patent Application Publication
(10) Pub. No.: US 2025/0345109 A1
(43) Pub. Date: Nov. 13, 2025

(54) POSITIONABLE, REMOVABLE SUCTION SYSTEM FOR USE DURING A SURGICAL PROCEDURE AND METHODS OF MAKING AND USING SAME

(71) Applicants: Mark McBride, Coronado, CA (US); Carola Romero, SanDiego, CA (US); Phong Duy Bui, San Diego, CA (US)

(72) Inventors: Mark McBride, Coronado, CA (US); Carola Romero, SanDiego, CA (US); Phong Duy Bui, San Diego, CA (US)

(21) Appl. No.: 19/236,380

(22) Filed: Jun. 12, 2025

Related U.S. Application Data
(63) Continuation-in-part of application No. 18/209,584, filed on Jun. 14, 2023.
Application No. 18/209,584 is a continuation-in-part of application No. 17/119,728, filed on Dec. 11, 2020, now abandoned.

(51) Int. Cl. A61B 18/00; A61B 46/20; A61B 46/23; A61B 90/00; A61B 90/30

(52) U.S. Cl. CPC A61B 18/00; A61B 46/23; A61B 90/08; A61B 90/30; A61B 90/39; A61B 2046/205; A61B 2090/309; A61B 2090/3966; A61B 2218/008

(57) ABSTRACT

The present invention is generally related to a suction system for use during surgical procedures. The suction system for use during surgical procedures utilizes a suction device that is postionable so that the suction device can be located in its most efficient position in order to capture as much smoke and odors as possible that are being produced at a surgical site. Furthermore, the suction system is also capable of being quickly and easily removed from the patient's surgical field so that the suction system can be quickly and easily located at another location on the patient's surgical field, if needed. Finally, the suction system includes widened head areas that create a wide suction area in order to further assist in capturing as much smoke and odors as possible that are being produced at a surgical site.

### CROSS-REFERENCE TO RELATED APPLICATIONS

This application is a continuation-in-part of U.S. patent application Ser. No. 18/209,584, filed on Jun. 14, 2023, which is a continuation-in-part of U.S. patent application Ser. No. 17/119,728, filed on Dec. 11, 2020, the disclosures of which are hereby incorporated by reference in their entirety to provide continuity of disclosures to the extent such disclosures are not inconsistent with the disclosure herein.

### FIELD OF THE INVENTION

The present invention is generally related to a suction system for use during surgical procedures. The suction system for use during surgical procedures of the present invention utilizes a suction device that is positionable so that the suction device can be located in its most efficient position in order to capture as much smoke and odors as possible that are being produced at a surgical site. Furthermore, the suction system of the present invention is also capable of being quickly and easily removed from the attachment at the margins of the surgical wound so that the suction system can be quickly and easily located at another location of the surgical field, if needed. Finally, the suction system includes widened head areas that create a wide suction area in order to further assist in capturing as much smoke and odors as possible that are being produced at a surgical site.

### BACKGROUND OF THE INVENTION

Prior to the present invention, as set forth in general terms above and more specifically below, it is known, to employ various types of suction systems for use during surgical procedures. Currently, a suction hose is conventionally connected to a conventional vacuum so that the suction hose is used to capture smoke and odors that are being produced at a surgical site. While these and other various suction systems for use during surgical procedures may have been generally satisfactory, there is nevertheless a need for a new and improved, positionable, removable suction system for use during surgical procedures.

It is a purpose of this invention to fulfill these and other needs in the art of suction system for use during surgical procedures in a manner more apparent to the skilled artisan once given the following disclosure.

The preferred positionable, removable suction system for use during surgical procedures, according to various embodiments of the present invention, offers the following advantages: ease of use; lightness in weight; durability; improved ability to remove the smoke and odors produced at a surgical site; the ability to position the suction system; the ability to easily remove the suction system; the ability to easily locate the suction system; increased suction area at the surgical site; the system is passive when positioned and does not require an assistant to continuously hold the device in place, and reduced noise produced by the suction system. In fact, in many of the preferred embodiments, these advantages are optimized to an extent that is considerably higher than heretofore achieved in prior, known suction systems for use during surgical procedures.

### BRIEF DESCRIPTION OF THE DRAWINGS

The above-mentioned features and steps of the invention and the manner of attaining them will become apparent, and the invention itself will be best understood by reference to the following description of the embodiments of the invention in conjunction with the accompanying drawings, wherein like characters represent like parts throughout the several views and in which:

FIG. 1 is a schematic, top view illustration of a positionable, removable suction system for use during surgical procedures, according to one embodiment of the present invention;

FIG. 2 is a schematic, bottom view illustration of the positionable, removable suction system for use during surgical procedures, according to one embodiment of the present invention;

FIGS. 3a-3c are schematic illustrations of the suction head of the positionable, removable suction system for use during surgical procedures, according to one embodiment of the present invention;

FIG. 4 is a schematic illustration of the suction head of the positionable, removable suction system for use during surgical procedures being removably attached to a surgical drape;

FIG. 5 is another schematic illustration of the suction head of the positionable, removable suction system for use during surgical procedures being removably attached to a surgical drape;

FIG. 6 is still another schematic illustration of the suction head of the positionable, removable suction system for use during surgical procedures being removably attached to a surgical drape; and

FIG. 7 is a schematic illustration of a re-locatable surgical drape pad, according to one embodiment of the present invention.

### DETAILED DESCRIPTION OF THE PREFERRED EMBODIMENTS OF THE INVENTION

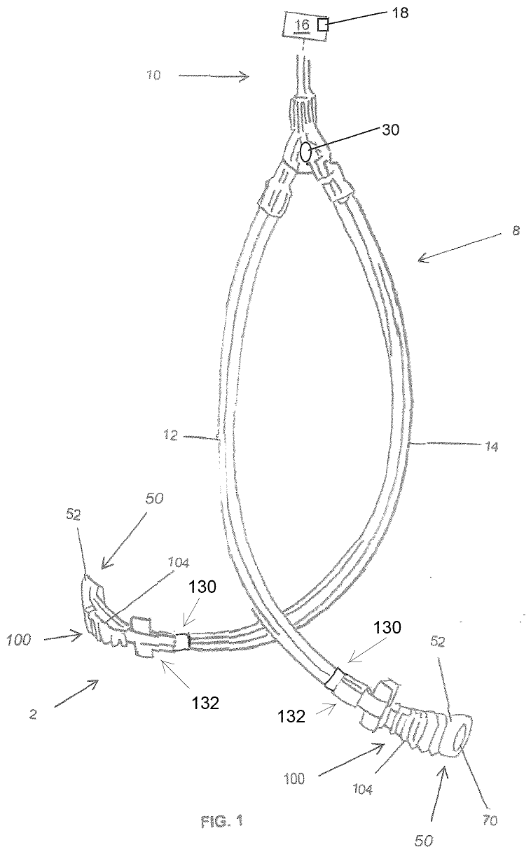

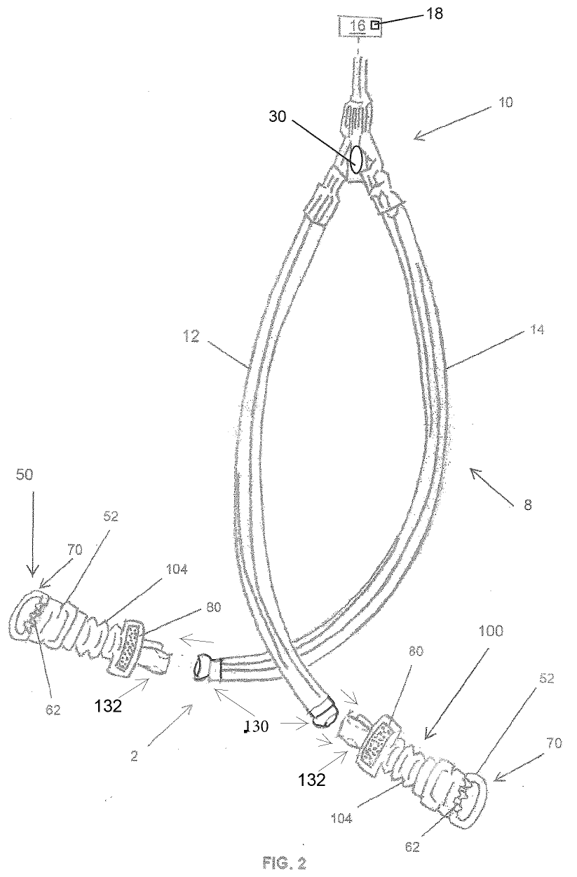

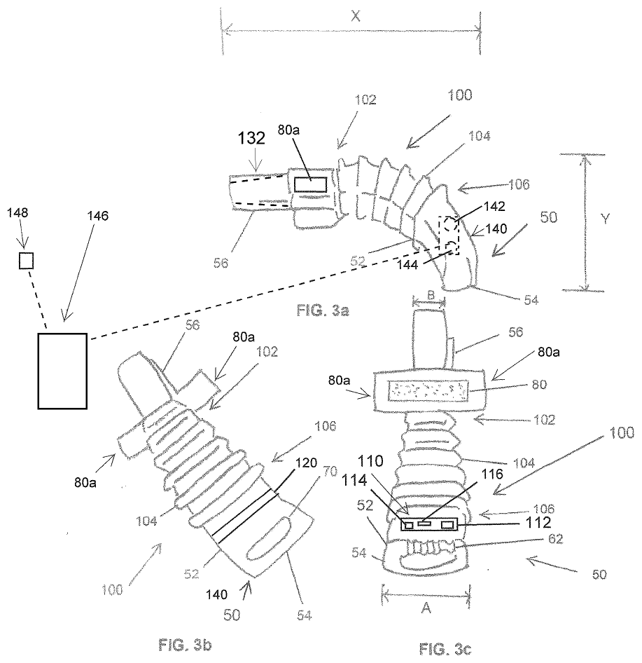

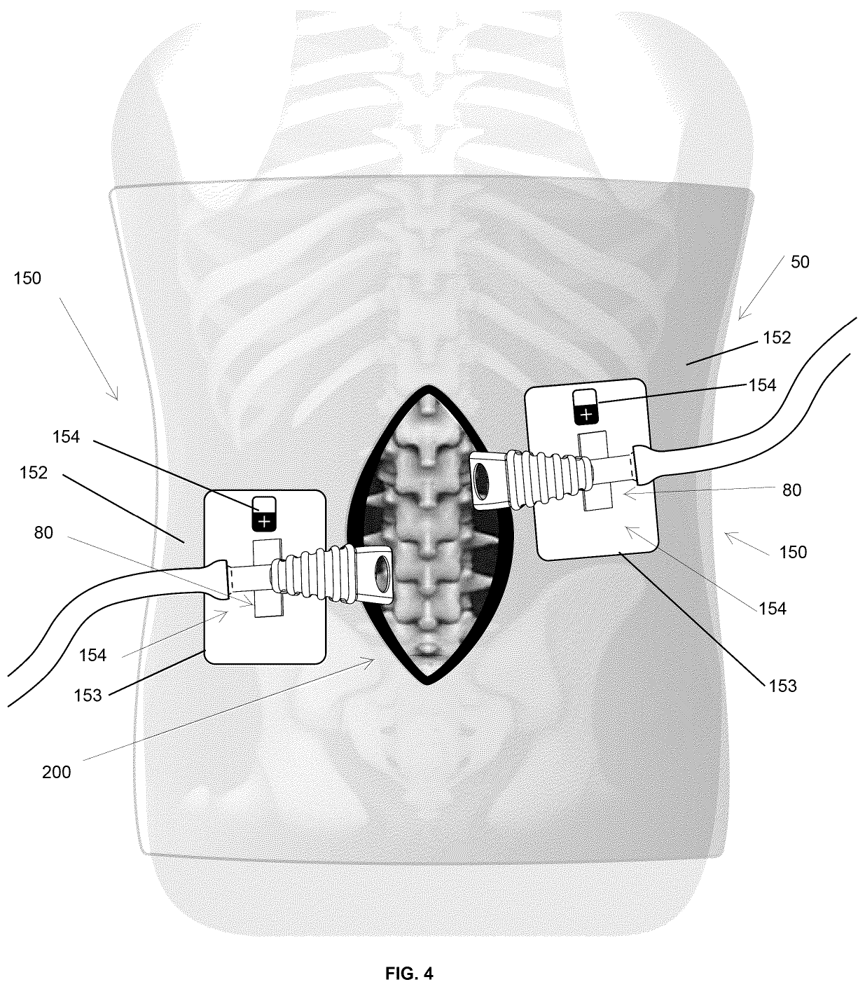

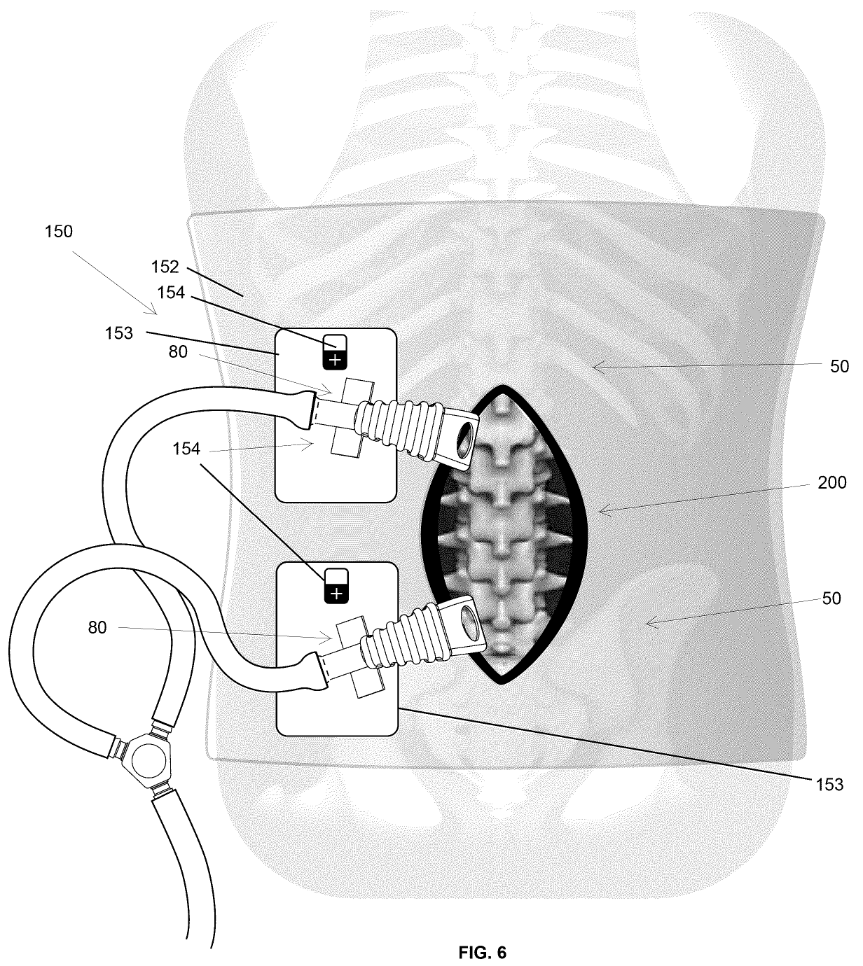

Referring now to FIGS. 1 and 2, there are illustrated a positionable, removable suction system 2 for use during surgical procedures. The positionable, removable suction system 2 for use during surgical procedures utilizes a suction device 50 that is positionable using a positioning device 100 (FIGS. 3a-6) so that the suction system 2 can be located in its most efficient position in order to capture as much smoke and odors as possible that are being produced at a surgical site 200 (FIGS. 4-6). Furthermore, the positionable, removable suction system 2 for use during surgical procedures is also capable of being quickly and easily removed from the surgical field so that the suction system 2 can be quickly and easily located at another location on the surgical field, if needed. Finally, the suction system 2 includes widened head areas in the suction device 50 that create a wide suction area in order to further assist in capturing as much smoke and odors as possible that are being produced at a surgical site.

With respect to positionable, removable suction system 2 for use during surgical procedures, suction system 2 includes a vacuum device 8 having vacuum source connector 10, suction hoses 12 and 14, and conventional vacuum source 16. Preferably, vacuum source connector 10 is constructed of any suitable, durable, high-strength, medical grade polymeric material. Preferably, suction hoses 12 and 14 are constructed of any suitable, durable, high-strength, flexible, medical grade polymeric material. Also, vacuum source connector 10 should be of such a size and length so that it is capable of adequately connecting to vacuum source 16 and suction hoses 12 and 14. Furthermore, suction hoses 12 and 14 should be of such a size and length so that they are capable of adequately providing the needed vacuum pressure to suction device 50 but also allow vacuum source 16 to be properly located away from the surgical procedure. It is to be understood that one end of each of the suction hoses 12 and 14 is conventionally connected to suction device 50, as will be described in greater detail later. Finally, it is to be understood that vacuum source 16 should be constructed so that it is able to provide the desired vacuum pressure to the suction device 50 so that suction device 50 is capable of capturing as much smoke and odors as possible that are being produced at a surgical site.

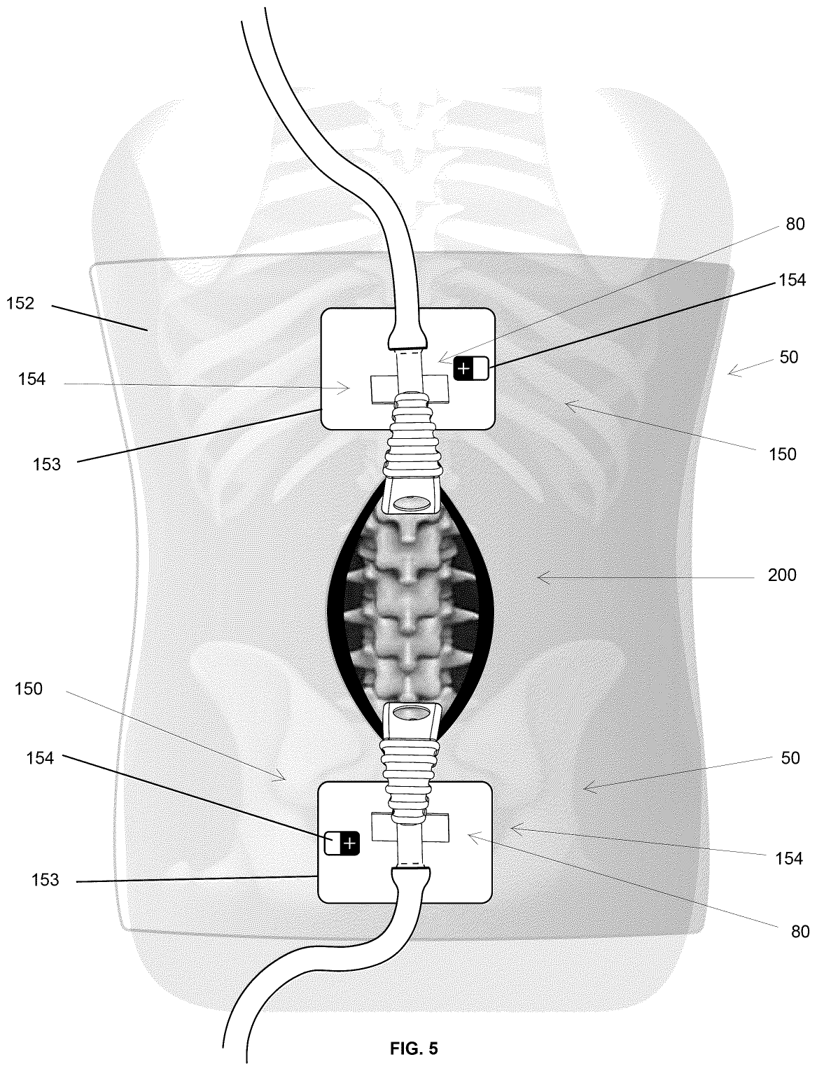

A unique aspect of the present invention is that the vacuum device 8 can be equipped with a vacuum controller 18. In one embodiment, the vacuum controller 18 can be configured to adjust the amount of vacuum air flow that is being drawn thorough the suction device 50. In this manner, if the surgical procedure creates a great amount of smoke and odor at the surgical site 200 (FIGS. 4 and 5), the vacuum controller 18 can be manipulated in order to increase the amount of vacuum air flow being drawn through the suction device 50 so that the smoke and odor can be more quickly removed from the surgical site 200. Conversely, the vacuum controller 18 can be manipulated in order to reduce the amount of vacuum air flow being drawn through the suction device 50, if necessary.

Another unique aspect of the present invention is that the vacuum device 8 can also be equipped with a vacuum flow diverter 30 located within the vacuum source connector 10. In one embodiment, the vacuum flow diverter 30 can be configured to be able to adjust the amount of vacuum air flow that is being drawn thorough an individual suction device 50 in positioning device 100. In this manner, if the surgical procedure creates a great amount of smoke and odor at one side of the surgical site 200 (FIGS. 4 and 5), the vacuum flow diverter 30 can be manipulated in order to increase the amount of vacuum air flow being drawn through a particular suction device 50 so that the smoke and odor can be more quickly removed from the surgical site 200.

For example, if there is a great amount of smoke and odor being produced at one side of the surgical site 200 (FIGS. 4 and 5), the vacuum flow diverter 30 can be manipulated in order to increase the amount of vacuum air flow being drawn through the suction device 50 that is located on that side of the surgical site 200 so that the smoke and odor can be more quickly removed from that side of the surgical site 200. It is to be understood that since more of the vacuum flow is being diverted to one of the suction devices 50, the amount of vacuum air flow going through the other suction device 50 will be accordingly reduced. Once the smoke and odor being produced at the one side of the surgical site 200 has been reduced or eliminated, the vacuum flow diverted 30 can then be manipulated so that the vacuum air flow being drawn through both suction devices 50 can be equalized back to a normal operating condition.

### Suction Device

As shown in FIGS. 3a-3c, positionable, removable suction system 2 for use during surgical procedures includes, in part, suction device 50. Suction device 50 includes, in part, suction head 52, suction head end 54, suction head connector 56, suction head ridges 62, suction head opening 70, and suction head fastener 80. It is to be understood that suction head 52, suction head end 54, suction head connector 56, and suction head ridges 62 are constructed of any suitable, durable, high strength, rigid, medical grade materials. It is to be understood that suction head connector 56 is used to conventionally connect suction head 52 to one end of each of the suction hoses 12, 14, as will be discussed in greater detail later. It is to be even further understood that suction head ridges 62 are conventionally formed along a length of each of the suction heads 52 at suction head end 54 by conventional techniques such as forming, molding, grinding or the like. Also, suction head 52, preferably, has a length of 1-2 cm along the direction of arrow (X) as shown in FIG. 3a and a curved distance (Y) of 1-2 cm as shown in FIG. 3a.

Another unique aspect of the present invention is that a width (A) of suction head 52 should be larger than a width (B) of suction head connector 56. In this manner, the wider size of section head 52 in comparison to section head connector 56 will further assist in capturing as much smoke and odors as possible that are being produced at a surgical site.

A unique aspect of the present invention is that that suction device 50 includes two (2) suction heads 52. Also, each suction device 50 is curved due to the corrugated construction of adjustable (malleable) memory section 104. In this manner, the use of the two (2) suction heads 52 and the curved nature of each of the suction device 50 allow suction devices 50 to be located on both sides of the surgical site 200 (FIGS. 4-6) in order to efficiently capture as much smoke and odors as possible that are being produced at a surgical site 200. Furthermore, the suction devices 50 create a passive suction system when positioned and the suction devices 50 do not require an assistant to continuously hold the suction devices 50 in place.

Another unique aspect of the present invention is the use of suction head ridges 62. As shown in FIGS. 1, 2, and 3c, the suction head ridges 62 are located along a length of the suction head end 54 in order to reduce the likelihood that suction head 52, in particular, suction head end 54 will create an undesirable seal on the area of the patient that is being operated upon if the suction head end 54 comes into close proximity with the area of the patient that is being operated upon.

With respect to FIGS. 1 and 3b, a further unique aspect of the present invention is illustrated. In particular, as shown in FIGS. 1 and 3b, suction head 52 also includes an opening 70. It is to be understood that opening 70 is formed in the top of suction head 52 by conventional techniques such as forming, cutting, drilling or the like. Opening 70 is used to reduce the vacuum noise that is created in suction head 52 by temporarily lowering the vacuum pressure at or near the suction head end 54. Furthermore, opening 70 makes it easier to vacuum more fragile things by reducing the vacuum pressure at or near the suction head end 54. Finally, opening 70 also acts as a safety valve, in that if a large item is accidentally vacuumed into suction head 52 and plugs suction head end 54, air will be drawn through opening 70 instead of burning out the motor in vacuum 16.

Suction Head Fastening Device

As shown in FIGS. 1, 2, 3c and 4-6, positionable, removable suction system 2 also includes suction head fasteners 80 and 80a. Preferably, fasteners 80 and 80a are constructed of any suitable hook and loop fastener, such as Velcro®. It is to be understood that fasteners 80 and 80a are attached to suction device 50 by conventional fastening techniques such as adhesives, tape, or the like.

With respect to suction head fasteners 80a, as shown in FIGS. 3a-3c, each positioning device 100 can be equipped with additional suction head fasteners 80a that can be attached to the sides of the suction device 50 adjacent to suction head fastener 80. It is to be understood that suction head fasteners 80a are attached to the sides of suction device 50 in a similar manner as suction head fastener 80. In this manner, if the positioning device 100 is oriented on its side, as shown in FIG. 2, the suction head fasteners 80a can be used to removably secure suction device 50 to a surgical drape 150 assembly (FIGS. 4-6) that is being used during a surgical procedure.

Another unique aspect of the present invention is the use of surgical drape fastener 154 (FIGS. 4-6). In particular, suction head fasteners 80 and 80a can be used to removably secure suction device 50 to a surgical drape 150 assembly (FIGS. 4-6) that is being used during a surgical procedure. As shown in FIGS. 4-6, the surgical drape assembly 150 includes a surgical drape 152 and a surgical drape pad 153 having a plurality of surgical drape fasteners 154. It is to be understood that the surgical drape fastener 154 is similar in construction to fasteners 80 and 80a. However, surgical fastener 154 is attached to surgical drape pad 153 in a similar manner that fasteners 80 and 80a are connected to suction device 50. As further shown in FIGS. 4-6, fasteners 80 and 80a and surgical drape fastener 154 are used in conjunction with each other to assist in retaining suction device 50 adjacent to the surgical field 200.

A still further unique aspect of the present invention is that fasteners 80 and 80a and fastener 154 allow suction device 50 to be easily and quickly removed from a particular location on the surgical field and, if needed, easily and quickly retained on another location on the surgical field as long as the surgical field has other similar hook and loop fasteners located on the surgical field at the other surgical sites. For example, if the patient is going to have several minor procedures performed during one long surgical procedure and those minor surgical procedures are located at different places on the patient, the surgical field can be equipped with similar hook and loop (Velcro®) fasteners located at the different surgical sites so that the suction device 50 can be moved from one surgical site 200 and located to and retained at the other surgical site 200 in a quick and easy manner. As will be discussed in greater detail later, in another embodiment, the surgical drape pads 153 can be configured to include an adhesive layer 156 (FIGS. 4-7) that will allow the surgical drape pad 153 to be removed from a current position, moved to another location on the surgical drape 152, and removably attached to that new location on the surgical drape 152.

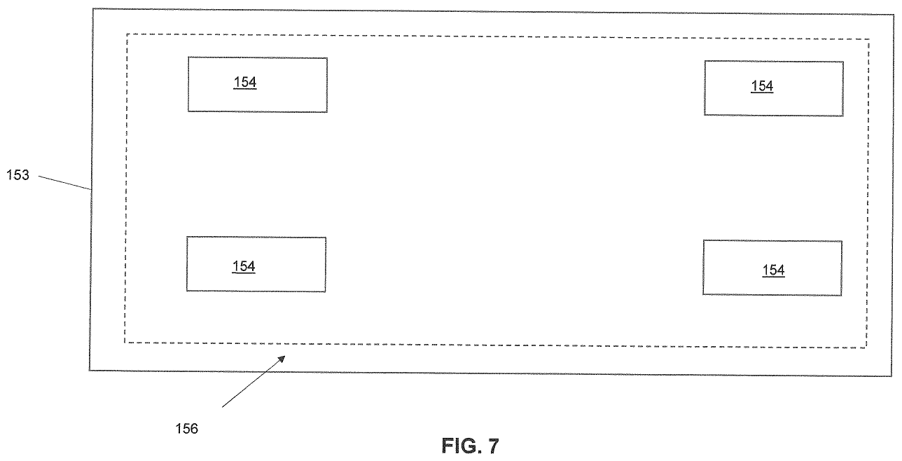

A yet another unique aspect of the present invention is the use of surgical drape pad 153. As shown in FIG. 7, surgical drape pad 153 is configured to be able to be removably attached to surgical drape 152 so that locations of the surgical pad 153 can be adjusted. For example, as shown in FIG. 4, the surgical drape pads 153 are located along the sides of the surgical site 200. However, if it is desired to perform another part of the surgical procedure on another part of the body, the surgical drape pads 153 can be moved to another location on the surgical drape 152 such as the top and bottom of the surgical site 200 (FIG. 5). The medical personnel performing the surgical procedure can simply pull up on the surgical drape pads 153 located in FIG. 4, relocate the surgical drape pads 153 to the new, desired locations, for example, as shown in FIG. 5, and press on the surgical drape pads 153 to secure the surgical drape pads 153 onto the surgical drape 152. In this manner, the suction devices 50 do not have to be removed and relocated to the new position. Only the surgical drape pads 153 need to be relocated. Since the suction devices 50 are already attached to the surgical drape pads 153, the suction devices 50 will automatically be moved to the new location on the surgical site 200.

As shown in FIG. 7, in order for the surgical pads 153 to be able to be removed from one location and secured at another location, the surgical drape pads 153 are configured with a plurality of fasteners 154 and an adhesive layer 156 that is conventionally applied to the bottom of the surgical pad 153. It is to be understood that adhesive layer 156 should be constructed of an adhesive that is capable of adequately securing the surgical pad 153 to the surgical drape 152, but is also allows the surgical pad 153 to be removed from the surgical drape 152, located to another area of the surgical drape 152, and then secured to that other area of the surgical drape 152.

### Positioning Device

With respect to FIGS. 3a-3c, there is illustrated positioning device 100. Positioning device 100 includes, in part, vacuum tube end 102, adjustable (malleable) memory section 104, and suction head end 106. It is to be understood that vacuum tube end 102 and suction head end 106, preferably, are constructed of any suitable, durable, high strength, rigid, medical grade materials. Also, it is to be understood that adjustable (malleable) memory section 104, preferably, is constructed of any suitable, durable, high strength, flexible, medical grade materials. It is to be further understood that a positioning device 100 is formed as part of each of the suction heads 52, as will be described in greater detail later.

Another unique aspect of the present invention is that adjustable (malleable) memory section 104 is to be constructed so that positioning device 100 will be able to bend and remain in the bent position in order to allow the suction heads 52 to be located within the surgical site 200 (FIGS. 4-6) at the desired locations, as will be discussed in greater detail later. In particular, adjustable (malleable) memory section 104 is constructed with a corrugated shape along the length of the adjustable (malleable) memory section 104. This corrugated shape allows for quick and easy adjustability of the positioning device 100. Furthermore, the corrugated shape allows the adjustable (malleable) memory section 104 to remain in the desired orientation throughout the surgical procedure.

### LED Light Assembly

Another unique aspect of the present invention is the use of a light emitting diode (LED) assembly 110. As shown in FIG. 3c, a LED assembly 110 can be conventionally attached to an outer surface of the suction head 52, adjacent to the front of the positioning device 100, and near the suction head end 54. It is to be understood that, in one embodiment, the LED assembly 110 includes, in part, a base 112 and a plurality of light emitting diodes (LEDs) 114 conventionally attached to the base 112. It is also to be understood that the LEDs 114 should be conventionally equipped with a power source 116 such as a battery. In this manner, the LED assembly 110 can be used to assist in illuminating the surgical site 200 (FIGS. 4 and 5).

### Radiopaque Strip

A still another unique aspect of the present invention is the use of a radiopaque strip 120 located on the positioning device 100 (FIG. 3b). In this manner, if the positioning device 100 inadvertently becomes dislodged from one of the suction hoses 12 or 14 and the dislodged positioning device 100 enters the surgical site 200 (FIGS. 4 and 5), a conventional X-ray device can be used to determine the location of the dislodged positioning device 100 so that the dislodged positioning device 100 can be removed from the surgical site 200.

### Rotatable Positioning Device

A yet another unique aspect of the present invention is that the positioning device 100 can rotate in relation to the suction hoses 12 and 14. In particular, as shown in FIG. 2, the positioning device 100 can rotate with respect to suction hoses 12 and 14. In one embodiment, a ball connector 130 is attached to the end of the suction hose 12 and 14. The ball connector 130 is configured to be able to interact with a rounded area 132 located within an interior surface of the suction head ends 56 in order to allow the ball connector 130 to rotate within the rounded area 132. In this manner, the positioning device 100 can be rotated to a desired orientation (as shown in FIGS. 1 and 2) so that the positioning device 100 is able in order to capture as much smoke and odors as possible that are being produced at a surgical site 200 (FIGS. 4 and 5).

### Vacuum Flow Blockage Detector

Another unique aspect of the present invention is use of a vacuum flow blockage detection assembly 140. As shown in FIG. 3a, in one embodiment, each suction device 50 is internally equipped with a vacuum flow blockage detection assembly 140 that is configured to assist in determining if there is a blockage within the suction device 50 of the positioning device 100 that is causing a reduction in the vacuum flow being drawn through the suction head end 54. For example, it may be possible that during a surgical procedure, body matter may be expelled from the surgical site 200 and become lodged in the suction device 50. In one embodiment, the vacuum flow blockage detection assembly 140 includes, in part, a vacuum flow sensor 142 and a transmitter 144. In particular, the vacuum flow sensor 142 is configured to be able to detect the amount of vacuum air flow that is being drawn into the suction device 50. The vacuum flow sensor 142 is also configured to interact with the transmitter 144 in order to transmit data to a processor 146. The processor 146 is configured to determine if the current amount of vacuum air flow that is being drawn through a suction device 50 falls within a threshold of a desired amount of vacuum air flow that should be drawn through the suction device 50. If the processor 146 determines that the current amount of vacuum air flow that is being drawn through a suction device 50 is not within the threshold of a desired amount of vacuum air flow that should be drawn through the suction device 50, the processor 144 is configured to send an alert to a medical personnel at the surgical site 200 (through a mobile computing device 148 associated with medical personnel such as a smartphone or the like) informing the system administrator at the surgical site 200 that there may be a blockage at the suction device 50. In this manner, the medical personnel can check the suction device 50 to determine, if there is a blockage at the suction device 50.

### Setting-Up the Positionable, Removable, Suction System

In order to set-up the positionable, removable suction system 2 for use during surgical procedures, attention is directed to FIGS. 1-7. The suction devices 50 are conventionally attached by suction head ends 56 to the suction hoses 12 and 14 through the use of ball connectors 130 and rounded areas 132, as discussed above.

Once each of the suction hoses 12, 14 has been connected to a suction device 50, suction head fasteners 80 and 80a are then placed on suction device 50. As discussed earlier, the suction head fasteners 80 and 80a and the surgical drape fastener 154 assist in retaining suction device 50 on the surgical drape 152. Also, the suction head fasteners 80 and 80a and the surgical drape fastener 154 can be used to removably, secure each of the suction devices 50 to the surgical field at locations proximate to the surgical site 200.

After the suction head fasteners 80 and 80a have been placed on each of the suction devices 50, each of the suction hoses 12, 14 are conventionally attached to the vacuum source 16 through the use of vacuum source connector 10, as discussed earlier.

### Operation of Positionable, Removable, Suction System

Another unique aspect of the present invention is the use of positionable, removable suction system 2 for use during surgical procedures. As shown in FIGS. 1-7, before the positionable, removable suction system 2 has been set-up, as discussed above, the surgical personnel determine where the surgical site 200 (or sites) are to be located on the patient. Once the surgical site 200 (or sites) has been determined, the surgical personnel set-up the positionable, removable suction system 2. The surgical personnel then conventionally attach surgical drape pads 152 having fasteners 154 to the surgical field/drape 152 around the surgical site 200. As discussed above, these fasteners 154 should be compatible with fasteners 80 and 80a on suction device 50. For example, the fasteners 154 located on the surgical drape pad 153 may be conventional hook and loop type fasteners (Velcro®) and the fasteners 80 and 80a on suction device 50 can also be constructed of a similar hook and loop type fastener. In this manner, suction head fasteners 80 and 80a can be quickly and easily secured to the fasteners 154 located adjacent to the surgical field at the desired surgical site 200 and then quickly and easily removed and placed at another surgical site 200 on the surgical field, if needed.

Once the fasteners 154 have been attached to the surgical drape pad 153 at the surgical site 200, the suction head fastener 80 or 80a can then be removably attached to the fasteners 154 on the surgical drape 152, as discussed above. The suction devices 50 can then be located/positioned using positioning device 100 so that they are located in their most efficient position in order to capture as much smoke and odors as possible that are being produced at a surgical site 200.

Another unique aspect of the present invention is the use of positioning device 100. As discussed above, positioning device 100 provides for the precise placement of the suction head 52 with respect to the surgical site 200 by allowing the surgical personnel to position each of the suction heads 52 so that each suction head 52 in its most efficient position in order to capture as much smoke and odors as possible that are being produced at a surgical site 200. The corrugated shape of the adjustable (malleable) memory section 104 provides the positioning device 100 with the ability to quickly and easily position the suction head 52 in a variety of positions and still be able to provide adequate vacuum pressure to the surgical site 200 in order to vacuum away the smoke and odors being emitted at the surgical site 200 during the surgical procedure.

As discussed above, a further unique aspect of the present invention is that a width (A) of suction head 52 should be larger than a width (B) of suction head connector 56. In this manner, the wider size of section head 52 in comparison to section head connector 56 will further assist in capturing as much smoke and odors as possible that are being produced at a surgical site.

As discussed earlier, once the surgical procedure has been completed at the surgical site 200 and there is a need to perform another surgical procedure at another surgical site 200 on the patient, surgical personnel turn off the vacuum source 16. The surgical personnel then simply remove the fastener 80 or 80a from the fasteners 154 on the surgical drape 152 at the current surgical site 200. The surgical personnel then locate the fastener 80 or 80a over the fasteners 154 on the surgical drape pad 153 at the next surgical site 200 on the surgical field and conventionally attach the fasteners 80 or 80a to the next set of fasteners 154 on the surgical drape pad 153 at the next surgical site 200. The surgical personnel then adjust the position of the suction heads 52 at the new surgical site 200 and turn the vacuum source 16 back on. The surgical personnel then can adjust, if needed, the position of the suction heads 52 using positioning device 100 with respect to the new surgical site 200 so that suction heads 52 are in their most efficient positions in order to capture as much smoke and odors as possible that are being produced at a surgical site 200.

As discussed above, instead of removing the suction heads 52 from the surgical drape pad 153, the surgical drape pad 153 can be removed from its current location, placed at another desired location on the surgical drape, and secured to the surgical drape 150. In this manner, the surgical heads 52 do not need to be removed from the surgical drape pad 153, moved to another location, and re-attached to the surgical drape 150 at the new location.

As discussed above, the LED assembly 110 can be used to assist in illuminating the surgical site 200 (FIGS. 4 and 5). Also, the radiopaque strip 120 and a conventional X-ray device can be used to determine the location of the dislodged positioning device 100 so that the dislodged positioning device 100 can be removed from the surgical site 200. Finally, the vacuum flow blockage detection assembly 140 can be used to assist in determining if there is a blockage within the suction device 50 of the positioning device 100 that is causing a reduction in the vacuum flow being drawn through the suction head end 54.

The preceding merely illustrates the principles of the invention. It will thus be appreciated that those skilled in the art will be able to devise various arrangements which, although not explicitly described or shown herein, embody the principles of the invention and are included within its spirit and scope. Furthermore, all examples and conditional language recited herein are principally intended expressly to be only for pedagogical purposes and to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention, as well as specific examples thereof, are intended to encompass both structural and functional equivalents thereof. Additionally, it is intended that such equivalents include both currently known equivalents and equivalents developed in the future, i.e., any elements developed that perform the same function, regardless of structure.

This description of the exemplary embodiments is intended to be read in connection with the figures of the accompanying drawing, which are to be considered part of the entire written description. In the description, relative terms such as “lower,” “upper,” “horizontal,” “vertical,” “above,” “below,” “up,” “down,” “top” and “bottom” as well as derivatives thereof (e.g., “horizontally,” “downwardly,” “upwardly,” etc.) should be construed to refer to the orientation as then described or as shown in the drawing under discussion. These relative terms are for convenience of description and do not require that the apparatus be constructed or operated in a particular orientation. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise.

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Thus, from the foregoing, it will be appreciated that, although specific embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims.

It is also to be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun. In addition, where features or aspects of the invention are described in terms of Markush groups, it is intended and those skilled in the art will recognize, that the invention embraces and is also thereby described in terms of any individual member or subgroup of members of the Markush group.

Therefore, provided herein is a new and improved positionable, removable suction system for use during surgical procedures. The preferred positionable, removable suction system for use during surgical procedures, according to various embodiments of the present invention, offers the following advantages: ease of use; lightness in weight; durability; improved ability to remove the smoke and odors produced at a surgical site; the ability to position the suction system; the ability to easily remove the suction system; the ability to easily locate the suction system; increased suction area at the surgical site; the ability to create a passive system when the suction devices are positioned in that the system does not require an assistant to continuously hold the suction devices in place; and reduced noise produced by the suction system.

In fact, in many of the preferred embodiments, these advantages of ease of use, lightness in weight, durability, improved ability to remove the smoke and odors produced at a surgical site, the ability to position the suction system, the ability to easily remove the suction system, the ability to easily locate the suction system, increased suction area at the surgical site, the ability to create a passive system when the suction devices are positioned in that the system does not require an assistant to continuously hold the suction devices in place, and reduced noise produced by the suction system are optimized to an extent that is considerably higher than heretofore achieved in prior, known suction system for use during surgical procedures.

We claim:

1. A positionable, removable, suction system for use during a surgical procedure, wherein the system comprises:
a plurality of suction hoses, wherein each of the plurality of suction hoses has a first end and a second end, and wherein each of the suction hoses includes a ball connector operatively connected to the first end of the suction hose;
a vacuum source connector directly connected to each of the second ends of the plurality of suction hoses;
a plurality of rotatable suctioning and positioning devices,
wherein each of the plurality of rotatable suctioning and positioning devices is directly connected to each of the first ends of the plurality of suction hoses at the ball connector,
wherein each of the plurality of rotatable suctioning and positioning devices is capable of being located on each side of a surgical procedure, and wherein each of the plurality of rotatable suctioning and positioning devices comprises;
a suction head connector having a first and second end, wherein the first end of the suction head connector includes a raised area that is configured to interact with the ball connector in order to allow the suction head connector to be rotatably connected to each of the first ends of the plurality of suction hoses,
a vacuum tube end having a first and second end, wherein the first end of the vacuum tube end is directly connected to the second end of the suction head connector,
a curved, corrugated adjustable memory section having a first and second end, wherein the first end of the curved, corrugated adjustable memory section is directly connected to the second end of the vacuum tube end, wherein the curved, corrugated adjustable memory section is constructed of a flexible, medical grade material,
a first suction head end, wherein the first suction head end is directly connected to the second end of the curved, corrugated adjustable memory section,
a suction head having a first and second end, wherein the first end of the suction head is directly connected to the first suction head end, and wherein the suction head includes an opening located on a top of the suction head,
a light assembly located on an outer surface of the suction head,
a second suction head end, wherein the second suction head end is directly connected to the second end of the suction head,
a plurality of suction head ridges located along a length of the second suction head end, and
a plurality of suction head fasteners attached to the second end of the suction head connector and the first end of the vacuum tube end; and
a surgical drape having a plurality of relocatable surgical drape pads, wherein each of the plurality of relocatable surgical pads includes a plurality of surgical drape pad fasteners, wherein each of the plurality of relocatable surgical drape pads are configured to be removably connected to the surgical drape, wherein the relocatable surgical drape pads are also configured to be able to be removed and then relocated on the surgical drape around a periphery of the surgical field when the surgical drape is located over a surgical field, and wherein the plurality of relocatable surgical drape pads are configured to be able to be located adjacent to the surgical field, and wherein each of the plurality of suction head fasteners is configured to be able to be removably connected to the surgical drape at least one of the plurality of surgical drape pad fasteners to assist in retaining each of the plurality of rotatable suctioning and positioning devices adjacent to the surgical field in order to capture as much smoke and odors as possible.

2. The positionable, removable, suction system, according to claim 1, wherein the system further comprises:
a vacuum source operatively connected to the vacuum source connector, wherein the vacuum source is configured to be able to adjust an amount of vacuum air flow that is being drawn thorough each of the plurality of suctioning and positioning devices.

3. The positionable, removable, suction system, according to claim 1, wherein each of the plurality of suction head fasteners further comprises:
a hook and loop fastener.

4. The positionable, removable, suction system, according to claim 1, wherein a width of the suction head is larger than a width of the suction head connector in order to assist in capturing as much smoke and odors as possible that are being produced at a surgical site.

5. The positionable, removable, suction system, according to claim 1, wherein each of the plurality of rotatable suctioning and positioning devices further comprises;
a radiopaque strip.

6. The positionable, removable, suction system, according to claim 1, wherein each of the plurality of rotatable suctioning and positioning devices further comprises;
a vacuum flow blockage detection assembly located within a portion of each of the plurality of rotatable suctioning and positioning devices, wherein the vacuum flow blockage detection assembly is configured to assist in determining if there is a blockage within the rotatable suctioning and positioning device that is causing a reduction in the vacuum flow being drawn through the rotatable suctioning and positioning device.

7. The positionable, removable, suction system, according to claim 1, wherein the vacuum source connector further comprises:
a vacuum flow diverter, wherein the vacuum flow diverter is configured to be able to adjust an amount of vacuum air flow that is being drawn thorough each of the plurality of rotatable suctioning and positioning devices.

8. A suction system, wherein the system comprises:
a plurality of suction hoses, wherein each of the plurality of suction hoses has a first end and a second end, and wherein each of the suction hoses includes a ball connector operatively connected to the first end of the suction hose;
a vacuum source connector directly connected to each of the second ends of the plurality of suction hoses;
a plurality of rotatable suctioning and positioning devices,
wherein each of the plurality of rotatable suctioning and positioning devices is directly connected to each of the first ends of the plurality of suction hoses at the ball connector,
wherein each of the plurality of rotatable suctioning and positioning devices is capable of being located on each side of a surgical procedure, and
wherein each of the plurality of rotatable suctioning and positioning devices comprises;
a suction head connector having a first and second end, wherein the first end of the suction head connector includes a raised area that is configured to interact with the ball connector in order to allow the suction head connector to be rotatably connected to each of the first ends of the plurality of suction hoses,
a vacuum tube end having a first and second end, wherein the first end of the vacuum tube end is directly connected to the second end of the suction head connector,
a curved, corrugated adjustable memory section having a first and second end, wherein the first end of the curved, corrugated adjustable memory section is directly connected to the second end of the vacuum tube end, wherein the curved, corrugated adjustable memory section is constructed of a flexible, medical grade material,
a first suction head end, wherein the first suction head end is directly connected to the second end of the curved, corrugated adjustable memory section,
a suction head having a first and second end, wherein the first end of the suction head is directly connected to the first suction head end, and wherein the suction head includes an opening located on a top of the suction head,
a light assembly located on an outer surface of the suction head,
a second suction head end, wherein the second suction head end is directly connected to the second end of the suction head,
a plurality of suction head ridges located along a length of the second suction head end, and
a plurality of suction head fasteners attached to the second end of the suction head connector and the first end of the vacuum tube end; and
a surgical drape having a plurality of relocatable surgical drape pads, wherein each of the plurality of relocatable surgical pads includes a plurality of surgical drape pad fasteners, wherein each of the plurality of relocatable surgical drape pads are configured to be removably connected to the surgical drape, wherein the relocatable surgical drape pads are also configured to be able to be removed and then relocated on the surgical drape around a periphery of the surgical field when the surgical drape is located over a surgical field, and wherein the plurality of relocatable surgical drape pads are configured to be able to be located adjacent to the surgical field, and wherein each of the plurality of suction head fasteners is configured to be able to be removably connected to the surgical drape at least one of the plurality of surgical drape pad fasteners to assist in retaining each of the plurality of rotatable suctioning and positioning devices adjacent to the surgical field in order to capture as much smoke and odors as possible.

9. The suction system, according to claim 8, wherein the system further comprises:
a vacuum source operatively connected to the vacuum source connector, wherein the vacuum source is configured to be able to adjust an amount of vacuum air flow that is being drawn thorough each of the plurality of suctioning and positioning devices.

10. The suction system, according to claim 8, wherein each of the plurality of suction head fasteners further comprises:
a hook and loop fastener.

11. The suction system, according to claim 8, wherein a width of the suction head is larger than a width of the suction head connector in order to assist in capturing as much smoke and odors as possible that are being produced at a surgical site.

12. The suction system, according to claim 8, wherein each of the plurality of rotatable suctioning and positioning devices further comprises;
a radiopaque strip.

13. The suction system, according to claim 8, wherein each of the plurality of rotatable suctioning and positioning devices further comprises;
a vacuum flow blockage detection assembly located within a portion of each of the plurality of rotatable suctioning and positioning devices, wherein the vacuum flow blockage detection assembly is configured to assist in determining if there is a blockage within the rotatable suctioning and positioning device that is causing a reduction in the vacuum flow being drawn through the rotatable suctioning and positioning device.

14. The suction system, according to claim 8, wherein the vacuum source connector further comprises:
a vacuum flow diverter, wherein the vacuum flow diverter is configured to be able to adjust an amount of vacuum air flow that is being drawn thorough each of the plurality of rotatable suctioning and positioning devices.

15. A method of constructing a positionable, removable, suction system for use during a surgical procedure, wherein the system comprises:
providing a plurality of suction hoses, wherein each of the plurality of suction hoses has a first end and a second end, and wherein each of the suction hoses includes a ball connector operatively connected to the first end of the suction hose;
attaching a vacuum source connector directly connected to each of the second ends of the plurality of suction hoses;
attaching a plurality of rotatable suctioning and positioning devices, wherein each of the plurality of rotatable suctioning and positioning devices is directly connected to each of the first ends of the plurality of suction hoses at the ball connector,
wherein each of the plurality of rotatable suctioning and positioning devices is capable of being located on each side of a surgical procedure, and
wherein each of the plurality of rotatable suctioning and positioning devices comprises;
a suction head connector having a first and second end, wherein the first end of the suction head connector includes a raised area that is configured to interact with the ball connector in order to allow the suction head connector to be rotatably connected to each of the first ends of the plurality of suction hoses,
a vacuum tube end having a first and second end, wherein the first end of the vacuum tube end is directly connected to the second end of the suction head connector,
a curved, corrugated adjustable memory section having a first and second end, wherein the first end of the curved, corrugated adjustable memory section is directly connected to the second end of the vacuum tube end, wherein the curved, corrugated adjustable memory section is constructed of a flexible, medical grade material,
a first suction head end, wherein the first suction head end is directly connected to the second end of the curved, corrugated adjustable memory section,
a suction head having a first and second end, wherein the first end of the suction head is directly connected to the first suction head end, and wherein the suction head includes an opening located on a top of the suction head,
a light assembly located on an outer surface of the suction head,
a second suction head end, wherein the second suction head end is directly connected to the second end of the suction head,
a plurality of suction head ridges located along a length of the second suction head end, and
a plurality of suction head fasteners attached to the second end of the suction head connector and the first end of the vacuum tube end; and
a surgical drape having a plurality of relocatable surgical drape pads, wherein each of the plurality of relocatable surgical pads includes a plurality of surgical drape pad fasteners, wherein each of the plurality of relocatable surgical drape pads are configured to be removably connected to the surgical drape, wherein the relocatable surgical drape pads are also configured to be able to be removed and then relocated on the surgical drape around a periphery of the surgical field when the surgical drape is located over a surgical field, and wherein the plurality of relocatable surgical drape pads are configured to be able to be located adjacent to the surgical field, and wherein each of the plurality of suction head fasteners is configured to be able to be removably connected to the surgical drape at least one of the plurality of surgical drape pad fasteners to assist in retaining each of the plurality of rotatable suctioning and positioning devices adjacent to the surgical field in order to capture as much smoke and odors as possible.

16. The method, according to claim 15, wherein the system further comprises:
attaching a vacuum source to the vacuum source connector, wherein the vacuum source is configured to be able to adjust an amount of vacuum air flow that is being drawn thorough each of the plurality of suctioning and positioning devices.

17. The method, according to claim 15, wherein a width of the suction head is larger than a width of the suction head connector in order to assist in capturing as much smoke and odors as possible that are being produced at a surgical site.

18. The method, according to claim 15, wherein each of the plurality of rotatable suctioning and positioning devices further comprises;
a radiopaque strip.

19. The method, according to claim 15, wherein each of the plurality of rotatable suctioning and positioning devices further comprises;
a vacuum flow blockage detection assembly located within a portion of each of the plurality of rotatable suctioning and positioning devices, wherein the vacuum flow blockage detection assembly is configured to assist in determining if there is a blockage within the rotatable suctioning and positioning device that is causing a reduction in the vacuum flow being drawn through the rotatable suctioning and positioning device.

20. The method, according to claim 15, wherein the vacuum source connector further comprises:
a vacuum flow diverter, wherein the vacuum flow diverter is configured to be able to adjust an amount of vacuum air flow that is being drawn thorough each of the plurality of rotatable suctioning and positioning devices.
